SEQUENCE LISTING (7 sequences)

SEQ ID NO: 1 - protein, 31 aa, Mycobacterium tuberculosis
DGSVVVNKVSELPAGHGLNVNTLSYGDLAAD

SEQ ID NO: 2 - protein, 16 aa, Mycobacterium tuberculosis
DGSVVVNKVSELPAGH

SEQ ID NO: 3 - protein, 15 aa, Mycobacterium tuberculosis
GLNVNTLSYGDLAAD

SEQ ID NO: 4 - protein, 15 aa, Mycobacterium tuberculosis
SELPAGHGLNVNLTS

SEQ ID NO: 5 - protein, 10 aa, Mycobacterium tuberculosis
DGSVVVNKVS

SEQ ID NO: 6 - protein, 10 aa, Mycobacterium tuberculosis
ELPAGHGLNV

SEQ ID NO: 7 - protein, 11 aa, Mycobacterium tuberculosis
NTLSYGDLAAD

(19) United States
(12) Patent Application Publication
(10) Pub. No.: US 2026/0115265 A1
(43) Pub. Date: Apr. 30, 2026

(54) METHODS AND COMPOSITIONS FOR PREVENTING OR TREATING FOOD ALLERGIES

(71) Applicant: Revolo Biotherapeutics Limited, Great Shelford, Cambridgeshire (GB)

(72) Inventors: Roly Foulkes, Great Shelford, Cambridgeshire (GB); Jorge De Alba, Great Shelford, Cambridgeshire (GB)

(21) Appl. No.: 18/866,048

(22) PCT Filed: May 16, 2023
(86) PCT No.: PCT/GB2023/051282

§ 371 Date: Nov. 14, 2024

Related U.S. Application Data
(60) Provisional application No. 63/342,454, filed on May 16, 2022.
(60) Provisional application No. 63/373,648, filed on Aug. 26, 2022.

(51) Int. Cl. A61K 38/52; A61P 37/08

(52) U.S. Cl. CPC A61K 38/52; A61P 37/08

(57) ABSTRACT

Described herein are methods for preventing or treating an food allergy or food intolerance by administrating a peptide related to Chaperonin 60.1.

### FIELD OF THE INVENTION

The embodiments of the present invention relate to compositions and methods for the prevention or treatment of food allergies and food intolerance with peptides related to Chaperonin 60.1 (Cpn60.1).

### BACKGROUND OF THE INVENTION

Food allergy is a costly, potentially life-threatening condition. It is a medical condition in which exposure to a food triggers a harmful immune response. Food allergy is increasingly recognized as a growing public health burden and has been referred to as the “second wave” of the allergy epidemic, following asthma.¹ Current evidence suggests that food allergies are common, affecting up to 10% of infants in some countries,² and have been increasing in prevalence in the last few decades. These increases in prevalence have preferentially affected industrialized regions, although there is now also growing evidence of increasing prevalence in rapidly developing countries commensurate with rising economic growth.³ In the United States, it is estimated that 32 million Americans have food allergies, including 5.6 million children under age 18⁴ Along with the U.S., Germany, Italy and Norway were reported to have the highest prevalence of food sensitivity—with about 22 percent of people from each country showing antibodies against some type of food.⁶

In view of the dramatic rise in the prevalence of food allergy globally, effective prevention strategies have become a public health priority. Several models have emerged around the etiology of food allergy, including the hygiene hypothesis, dual allergen exposure hypothesis, and vitamin D hypothesis. Other nutritional interventions, including the use of whey-based, partially hydrolyzed formula in non-breastfed infants, also play an important role. In recent years, there has been a shift away from prolonged food allergen avoidance to the proactive allergen introduction from 4 months of age. However, the implementation of this strategy at the population level still raises significant logistic problems, including patient selection and development of suitable food formats for young infants. The treatment of food allergies has also seen major transformations. While, there is a greater focus on desensitization and tolerance induction by oral and epicutaneous immunotherapy. In addition, specialized hypoallergenic infant formulas for the treatment of infants with cow's milk allergy have undergone reformulation, including the addition of lactose and probiotics in order to modulate the gut microbiome and early immune responses. For a number of reasons, these strategies are inadequate and strict allergen avoidance remains the key prevention and treatment principle.

Accordingly, there is a need for effective compositions and methods for the prevention or treatment of food allergies and food intolerance.

### BRIEF SUMMARY OF THE INVENTION

The embodiments of the present invention provide a method for treating or preventing the onset of a food allergy or food intolerance to a dietary antigen derived from a food in a subject in need thereof by administering to the subject, a peptide related to Chaperonin 60.1 (Cpn60.1). In some embodiments, the Cpn60.1-related peptide is selected from: DGSVVVNKVSELPAGHGLNVNTLSYGDLAAD (SEQ ID NO: 1) (PIN201104); DGSVVVNKVSELPAGH (SEQ ID NO: 2); GLNVNTLSYGDLAAD (SEQ ID NO: 3); SELPAGHGLNVNLTS (SEQ ID NO: 4); DGSVVVNKVS (SEQ ID NO: 5); ELPAGHGLNV (SEQ ID NO: 6); NTLSYGDLAAD (SEQ ID NO: 7); or a functionally equivalent fragment or variant thereof. In one embodiment, the Cpn60.1-related peptide is SEQ ID NO:1.

In some embodiments, the method further comprises, prior to administration of the Cpn60.1-related peptide, diagnosing the subject as having, or likely to develop, a food allergy or food intolerance, or receiving the results of an assay that diagnoses the subject as having, or likely to develop, a food allergy or food intolerance.

In some embodiments, the Cpn60.1-related peptide is administered prior to the first exposure to a potential causal food allergen. In alternate embodiments, the Cpn60.1-related peptide is administered upon clinical signs of atopic symptoms.

In some embodiments, the Cpn60.1-related peptide is administered to a subject that has been diagnosed with at least one food allergy or food intolerance to a dietary antigen. In some embodiments, the dietary antigen is derived from milk and products thereof; eggs and products thereof; meat and products thereof; fish, mollusks, and crustaceans and products thereof; oils, fats, and products thereof; grains and products thereof; pulses, seeds, kernels, nuts, and products thereof; vegetables and products thereof; fruits and products thereof; mushrooms and products thereof; sugar, sugar products, chocolate products, and confectionary; and spices and herbs.

Other implementations are also described and recited herein.

### BRIEF DESCRIPTION OF THE DRAWINGS

For the purpose of illustration, certain embodiments of the present invention are shown in the drawings described below. It should be understood, however, that the invention is not limited to the precise arrangements, dimensions, and instruments shown. In the drawings:

FIG. 1 shows a diagram of the two phases associated with an allergic inflammation reaction: early and late phase responses. Cpn60.1-related peptides have previously been shown to be very effective in blocking the inflammation associated with the late phase response. The effects on inflammatory cells last for more than 14 days (eosinophils, neutrophils, lymphocytes and cytokines). Abbreviations: allergen-induced early asthmatic response (EAR); allergen-induced late asthmatic response (LAR); forced expiratory volume in 1 second (FEV1); inhaled corticostereoid (ICS); short-acting inhaled β2-agonists (SABA).

FIG. 2 illustrates the design protocol and endpoints of the study assessing the effects of 1140 (80 or 160 g/kg; three or six doses) three days after the last dose using in an ovalbumin-induced mouse model of food allergy.

FIG. 3 shows the effects of prophylactic and therapeutic dosing with '1104 (80 or 160 g/kg; three or six doses) on the severity of food allergy in an ovalbumin-induced food allergy mouse model as measured on a clinical scoring scale. Data are expressed as mean±SEM; n=8-16. Comparisons to the OVA vehicle group was made using a one-way analysis of variance (ANOVA), followed by a Dunnett's test. *P<0.05, **P<0.01, and ***P<0.001.

FIG. 4 shows that prophylactic and therapeutic dosing with '1104 (80 or 160 g/kg; three or six doses) significantly reduced four measures of food allergy at day 28, three days after the last dose of '1104 in an ovalbumin-induced food allergy mouse model: OVA-specific immunoglobulin E (IgE) (upper left panel); murine mast cell protease (mMCP-1) (upper right panel); body temperature (bottom left panel); and clinical scoring (bottom right panel). Data are expressed as mean±SEM; n=8-16. Comparisons to the OVA vehicle group was made using an ANOVA, followed by a Dunnett's test. *P<0.05, **P<0.01, and ***P<0.001.

FIG. 5 shows that prophylactic and therapeutic dosing with '1104 (80 or 160 g/kg; three or six doses) significantly reduced key Th2 cytokines/chemokines levels in the serum in an ovalbumin-induced food allergy mouse model: IL-4 (upper left panel); IL-5 (upper right panel); IL-13 (bottom left panel); and Eotaxin (bottom right panel). Each column represents the mean, and each bar represents the standard error of the mean of n=8-16. OVA+vehicle was compared to each of the other treatment groups using an ANOVA followed by Dunnett's test. *P<0.05, **P<0.01 and *** P<0.001.

FIG. 6 illustrates the design protocol and endpoints of the study assessing the effects of '1104 (80 or 800 g/kg; six doses) three and 13 days after the last dose using in an ovalbumin-induced mouse model of food allergy.

FIG. 7 shows the effects of '1104 (80 or 800 g/kg; six doses) on the severity of food allergy in an ovalbumin-induced food allergy mouse model as measured on a clinical scoring scale three and 13 days after the last dose of '1104. Data are expressed as mean±SEM; n=8. Comparisons to the OVA vehicle group was made using an ANOVA, followed by a Dunnett's test. *P<0.05, **P<0.01, and ***P<0.001.

FIG. 8 shows that '1104 (80 or 800 g/kg; six doses) significantly reduced two measures of food allergy at day 28, three days after the last dose of '1104 (left panels) and 13 days after the last dose of '1104 (right panels) in an ovalbumin-induced food allergy mouse model: clinical symptom scoring (upper panels) and body temperature (bottom panels). Data are expressed as mean±SEM; n=8. Comparisons to the OVA vehicle group was made using an ANOVA, followed by a Dunnett's test. *P<0.05 and ***P<0.001.

FIG. 9 shows that '1104 (80 or 800 g/kg; six doses) significantly reduced two measures of food allergy at day 28, three days after the last dose of '1104 (left panels) and 13 days after the last dose of '1104 (right panels) in an ovalbumin-induced food allergy mouse model: OVA-specific IgE (upper panels) and mMCP-1 (bottom panels). Data are expressed as mean±SEM; n=8. Comparisons to the OVA vehicle group was made using an ANOVA, followed by a Dunnett's test. *P<0.05, **P<0.01, and ***P<0.001.

FIG. 10 shows that '1104 (80 or 800 g/kg; six doses) significantly reduced key Th2 cytokines/chemokines levels in the serum at day 28, three days after the last dose of '1104 (left panels) and 13 days after the last dose of '1104 (right panels) in an ovalbumin-induced food allergy mouse model: IL-4 (upper panels) and IL-5 (bottom panels). Data are expressed as mean±SEM; n=8. Comparisons to the OVA vehicle group was made using an ANOVA, followed by a Dunnett's test. *P<0.05, **P<0.01, and ***P<0.001.

FIG. 11 shows that '1104 (80 or 800 g/kg; six doses) significantly reduced key Th2 cytokines/chemokines levels in the serum at day 28, three days after the last dose of '1104 (left panels) and 13 days after the last dose of '1104 (right panels) in an ovalbumin-induced food allergy mouse model: IL-13 (upper panels) and Eotaxin (bottom panels). Data are expressed as mean±SEM; n=8. Comparisons to the OVA vehicle group was made using an ANOVA, followed by a Dunnett's test. **P<0.01 and ***P<0.001.

FIG. 12 shows that '1104 (80 or 800 g/kg; six doses) significantly reduced key cytokines/chemokines levels in the serum at day 28, three days after the last dose of '1104 (left panels) and 13 days after the last dose of '1104 (right panels) in an ovalbumin-induced food allergy mouse model: IL-9 (upper panels) and IL-10 (bottom panels). Data are expressed as mean±SEM; n=8. Comparisons to the OVA vehicle group was made using an ANOVA, followed by a Dunnett's test. **P<0.01 and ***P<0.001.

FIG. 13 shows that '1104 (80 or 800 g/kg; six doses) significantly reduced key cytokines/chemokines levels in the serum at day 28, three days after the last dose of '1104 (left panels) and 13 days after the last dose of '1104 (right panels) in an ovalbumin-induced food allergy mouse model: IL-17 (upper panels) and CCL-17 (bottom panels). Data are expressed as mean±SEM; n=8. Comparisons to the OVA vehicle group was made using an ANOVA, followed by a Dunnett's test. *P<0.05 and ***P<0.001.

FIG. 14 shows that '1104 (80 or 800 g/kg; six doses) significantly reduced key cytokines/chemokines levels in the serum at day 28, three days after the last dose of '1104 (left panels) and 13 days after the last dose of '1104 (right panels) in an ovalbumin-induced food allergy mouse model: CCL-22 (upper panels) and IFN (bottom panels). Data are expressed as mean±SEM; n=8. Comparisons to the OVA vehicle group was made using an ANOVA, followed by a Dunnett's test. *P<0.05 and ***P<0.001.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two-standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex.

As used herein, the terms “effective amount” and “therapeutically-effective amount” include an amount sufficient to prevent or ameliorate a manifestation of disease or medical condition, such as an immune disorder including eosinophilic esophagitis, hemolytic anemia, thrombocytopenia, thyroiditis, pernicious anemia, Addison's disease, autoimmune diabetes, myasthenia gravis, rheumatoid arthritis, systemic lupus erythematosus, atherosclerosis, and autoimmune encephalitis, allergic conditions such as eczema, dermatitis, allergic rhinitis, allergic conjunctivitis, allergic airways diseases, hyper-eosinophilic syndrome, contact dermatitis, food allergy, and respiratory diseases characterized by eosinophilic airway inflammation and airway hyper-responsiveness, such as allergic asthma, intrinsic asthma, allergic bronchopulmonary aspergillosis, eosinophilic pneumonia, allergic bronchitis bronchiectasis, occupational asthma, reactive airway disease syndrome, interstitial lung disease, hyper-eosinophilic syndrome, or parasitic lung disease. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treating” or “treatment” or “to treat” or “alleviating” or “to alleviate” refer to both (1) therapeutic measures that cure, slow down, lessen symptoms of, and/or halt progression of a diagnosed disease or infection and (2) prophylactic or preventative measures that prevent or slow the development of a disease or infection.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of an immune disorder, delay or slowing of an immune disorder, and an increased lifespan as compared to that expected in the absence of treatment.

As used herein, the term “short-term administration” or “acute administration” means that the therapeutic agent or drug is administered as one dose or daily doses for a period of 2, 3, 4, 5, 6, 7 days or more. The “short-term administration” can be administered as a prophylactic measure prior to potential exposure to one or more causal food allergens or after possible exposure to one or more causal food allergens, or therapeutically after the onset of symptoms after exposure to one or more causal food allergens.

As used herein, the term “long-term administration” means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

The terms “decrease,” “reduced,” “reduction,” or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, a nucleic acid encoding a polypeptide as described herein (e.g., an antibody or antibody reagent) is comprised by a vector. In some of the aspects described herein, a nucleic acid sequence encoding a given polypeptide as described herein, or any module thereof, is operably linked to a vector. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion thereof, or chimeric antigen receptor (CAR) described herein is isolated. In some embodiments, the antibody, antibody reagent, antigen-binding portion thereof, or CAR described herein is purified.

As used herein, “engineered” refers to the aspect of having been manipulated by the hand of man. For example, an antibody, antibody reagent, antigen-binding portion thereof, CAR or bispecific antibody is considered to be “engineered” when the sequence of the antibody, antibody reagent, antigen-binding portion thereof, CAR or bispecific antibody is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher et al. (1996).⁷

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;⁸ The Encyclopedia of Molecular Cell Biology and Molecular Medicine;⁹ Molecular Biology and Biotechnology: a Comprehensive Desk Reference;¹⁰ Immunology;¹¹ Janeway's Immunobiology;¹² Lewin's Genes XI;¹³ Molecular Cloning: A Laboratory Manual;¹⁴ Basic Methods in Molecular Biology;¹⁵ Laboratory Methods in Enzymology;¹⁶ Current Protocols in Molecular Biology (CPMB);¹⁷ Current Protocols in Protein Science (CPPS);¹⁸ and Current Protocols in Immunology (CPI).¹⁹

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### Food Allergies

An allergy is an immune malfunction in which an individual is hypersensitized to react immunologically to typically perse harmless substances called allergens. The principal antibody involved in allergic reactions is immunoglobulin E (IgE). IgE has an essential role in type I hypersensitivity,²⁰ which manifests in various allergic diseases, such as allergic asthma, most types of sinusitis, allergic rhinitis, food allergies, and specific types of chronic urticaria and atopic dermatitis. IgE also plays a pivotal role in responses to allergens, such as: anaphylactic reactions to drugs, bee stings, and antigen preparations used in desensitization immunotherapy.

The constant region (Fc region) of IgE is able to bind to specific receptors of cells, which are able to release histamine or other inflammatory mediators, cytokines and/or proteases into the surrounding tissue. Histamine-releasing cells are mainly mast and basophilic cells. The release of histamine is initiated when cell-bound IgE is contacted and cross-linked by the allergen. Histamine, which is stored mainly in mast cells and basophils, is a prominent contributor to allergic disease. Elevations in plasma or tissue histamine levels have been noted during anaphylaxis and experimental allergic responses of the skin, nose, and airways. Histamine released in the nose, eyes, and sinuses, for example, stimulates sneezing, a runny nose, and itchy eyes; released in the lungs, it causes narrowing and swelling of the lining of the airways and the secretion of thick mucus; in the skin, rashes and hives; and in the digestive system, stomach cramps and diarrhea. Typical allergens are derived from plant pollens (e.g., rye grass, ragweed, timothy grass, birch trees pollens), mold spores, drugs (e.g., penicillins, sulfonamides, salicylates and local anesthetics), foods (e.g., nuts, seafood, egg, peas, beans, peanuts and other legumes, milk), insect products (e.g., bee-sting venom, wasp sting venom, cockroach calyx, dust mites), and animal hair and dander.

A food allergy is an abnormal immune response to food. The signs and symptoms may range from mild to severe, and may include itchiness, swelling of the tongue, vomiting, diarrhea, hives, trouble breathing, or low blood pressure, and, when severe, anaphylaxis. This typically occurs within minutes to several hours of exposure. Although sensitivity levels vary by country, the most common food allergies are allergies to milk, eggs, peanuts, tree nuts, seafood, shellfish, soy, and wheat. One of the most common food allergies is a sensitivity to peanuts, a member of the bean family. Peanut allergies may be severe, but children with peanut allergies sometimes outgrow them. Tree nuts, including cashews, Brazil nuts, hazelnuts, macadamia nuts, pecans, pistachios, pine nuts, coconuts, and walnuts, are also common allergens. Sufferers may be sensitive to one particular tree nut or to many different ones. Furthermore, seeds, including sesame seeds and poppy seeds, contain oils where protein is present, which may elicit an allergic reaction.

Diagnosis is usually based on a medical history, elimination diet, skin prick test, blood tests for food-specific IgE antibodies, or oral food challenge. For skin-prick tests, a tiny board with protruding needles is used. The allergens are placed either on the board or directly on the skin. The board is then placed on the skin, to puncture the skin and for the allergens to enter the body. If a hive appears, the person is considered positive for the allergy. This test only works for IgE antibodies. Allergic reactions caused by other antibodies cannot be detected through skin-prick tests. Patch testing is used to determine if a specific substance causes allergic inflammation of the skin. It tests for delayed food reactions. Blood testing is another way to test for allergies; however, it poses the same disadvantage and only detects IgE allergens and does not work for every possible allergen. Food challenges test for allergens other than those caused by IgE allergens. The allergen is given to the person in the form of a pill, so the person can ingest the allergen directly. The person is watched for signs and symptoms. The problem with food challenges is that they must be performed in the hospital under careful watch, due to the possibility of anaphylaxis. For tests that involve a reaction of the subjects themselves, subjects cannot be administered many different tests in a short period of time. In addition, these types of tests are expensive and invasive. Additional diagnostic tools for evaluation of eosinophilic or non-IgE antibody mediated reactions include endoscopy, colonoscopy, and biopsy.

### Chaperonin 60.1-Related Peptides

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Compositions useful in the methods of the present invention include, but are not limited to:

(SEQ ID NO: 1)
DGSVVVNKVSELPAGHGLNVNTLSYGDLAAD (PIN201104);
(SEQ ID NO: 2)
DGSVVVNKVSELPAGH;
(SEQ ID NO: 3)
GLNVNTLSYGDLAAD;
(SEQ ID NO: 4)
SELPAGHGLNVNLTS;
(SEQ ID NO: 5)
DGSVVVNKVS;
(SEQ ID NO: 6)
ELPAGHGLNV;
(SEQ ID NO: 7)
NTLSYGDLAAD;

or a functionally equivalent fragment or variant thereof.

Other compositions useful in the methods of the present invention include, but are not limited to, Cpn60.1-related peptides disclosed in United States Published Patent Application No. 20040132163²¹ and U.S. Pat. Nos. 11,098,090;²² 9,320,791;²³ 9,085,632.24

In certain aspects, a Cpn60.1-related peptide is composed of amino acid residues. As used herein, the term “amino acid residue” is used interchangeably with the terms “amino acid” or “aa” to refer to an amino acid which is part of a peptide or protein. In some such aspects, an agonist or ligand of the present invention is composed of amino acids with the standard structure NH2-C(H)(R)—COOH, where R represents an individual amino acid side chain. In certain aspects, an agonist or ligand is composed of amino acid residues which are naturally occurring amino acids. In certain aspects, a naturally occurring amino acid includes one of the twenty standard amino acids found in naturally occurring peptides and proteins. In some such aspects, an agonist or ligand is composed of at least one naturally occurring amino acid residue which is alanine (“A”), arginine (“R”), asparagine (“N”), aspartic acid (“D”), cysteine (“C”), glutamine (“Q”), glutamic acid (“E”), glycine (“G”), histidine (“H”), isoleucine (“I”), leucine (“L”), lysine (“K”), methionine (“M”), phenylalanine (“F”), proline (“P”), serine (“S”), threonine (“T”), tryptophan (“W”), tyrosine (“Y”), or valine (“V”).

In other aspects a Cpn60.1-related peptide used in the methods of the present invention is composed of at least one amino acid residue which is an unnatural or synthetic amino acid. In some such aspects, an unnatural or synthetic amino is a chemically modified amino acid including but not limited to amino acids which have been modified by methylation, amidation, acetylation, protecting groups, and/or substitution with other chemical groups that can change the physiochemical properties of a peptide. In further aspects, an unnatural or synthetic amino is a chemically modified amino acid which has been modified with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, and the like).

In further aspects, a Cpn60.1-related peptide used in the methods of the present invention is composed of at least one amino acid which has an L-configuration (the chirality of an L-amino acid). In additional aspects, a Cpn60.1-related peptide used in the methods of the present invention is composed of at least one amino acid which has a D-configuration (the chirality of a D-amino acid).

In certain aspects, an agonist or ligand of the present invention is about 50 aa in length; 49 aa in length; 48 aa in length; 47 aa in length; 46 aa in length; 45 aa in length; 44 aa in length; 43 aa in length; 42 aa in length; 41 aa in length; 40 aa in length; 39 aa in length; 39 aa in length; 38 aa in length; 37 aa in length; 36 aa in length; 35 aa in length; 34 aa in length; 33 aa in length; 32 aa in length; 31 aa in length; 30 aa in length; 29 aa in length; 28 aa in length; 27 aa in length; 26 aa in length; 25 aa in length; 24 aa in length; 23 aa in length; 22 aa in length; 21 aa in length; or 20 aa in length; 19 aa in length; 18 aa in length; 17 aa in length; 16 aa in length; 15 aa in length; 14 aa in length; 13 aa in length; 12 aa in length; 11 aa in length; 10 aa in length; 9 aa in length; 8 aa in length; 7 aa in length; 6 aa in length; or 5 aa in length.

In certain aspects, compositions of the present invention are administered to a patient by any appropriate route known and/or employed by those skilled in the art. In some aspects, compositions of the present invention are administered by oral (PO), intravenous (IV), intramuscular (IM), intra-arterial, intramedullary, intrathecal, subcutaneous (SQ), intraventricular, transdermal, interdermal, intradermal, rectal (PR), vaginal, intraperitoneal (IP), intragastric (IG), topical (e.g., by powders, ointments, creams, gels, lotions, and/or drops), mucosal, intranasal, buccal, enteral, intravitreal, sublingual, by intratracheal instillation, bronchial instillation, and/or inhalation, as an oral spray, nasal spray, aerosol, and/or through a portal vein catheter.

In certain aspects, a Cpn60.1-related peptide used in the methods of the present invention and/or pharmaceutical compositions thereof may be administered intravenously, for example, by intravenous infusion. In further aspects, a Cpn60.1-related peptide used in the methods of the present invention and/or pharmaceutical compositions thereof may be administered by intramuscular injection. In more aspects, a Cpn60.1-related peptide used in the methods of the present invention and/or pharmaceutical compositions thereof may be administered by intratumoral injection. In certain aspects, a Cpn60.1-related peptide used in the methods of the present invention and/or pharmaceutical compositions thereof may be administered by subcutaneous injection. In further aspects, a Cpn60.1-related peptide used in the methods of the present invention and/or pharmaceutical compositions thereof may be administered via portal vein catheter. In more aspects, the invention encompasses the delivery of a Cpn60.1-related peptide used in the methods of the present invention and/or pharmaceutical compositions thereof by any appropriate route taking into consideration likely advances in the art of drug delivery.

In some aspects, a Cpn60.1-related peptide used in the methods of the present invention and/or pharmaceutical compositions thereof may be administered at dosage levels sufficient to deliver from about 0.001 mg/kg to 100 mg/kg, from about 0.01 mg/kg to 50 mg/kg, from about 0.1 mg/kg to 40 mg/kg, from about 0.5 mg/kg to 30 mg/kg, from about 0.01 mg/kg to 10 mg/kg, from about 0.1 mg/kg to 10 mg/kg, or from about 1 mg/kg to 25 mg/kg of patient body weight per day to obtain the desired therapeutic effect. In certain aspects, the desired dosage may be delivered more than three times per day, three times per day, two times per day, once per day, once every other day, once every third day, once every week, once every two weeks, once every three weeks, once every four weeks, once every two months, once every six months, or once every twelve months. In certain aspects, the desired dosage may be delivered using multiple administrations (e.g., two, three, four, five, six, seven, eight, nine, ten, eleven, twelve, thirteen, fourteen, or more administrations). In certain aspects, the desired dosage may be delivered using one or more administrations during an initial period of time, followed by a period of time in which no dosage is administered.

In further aspects, a Cpn60.1-related peptide used in the methods of the present invention may be utilized for prophylactic applications. In more aspects, prophylactic applications involve systems and methods for preventing, inhibiting progression of, and/or delaying the onset of a food allergy, in individuals susceptible to and/or displaying symptoms of the food allergy.

In certain aspects, a Cpn60.1-related peptide used in the methods of the present invention is administered to a target cell in vivo. In other aspects, a Cpn60.1-related peptide used in the methods of the present invention is administered to a target cell ex vivo. In additional aspects, a Cpn60.1-related peptide used in the methods of the present invention is administered to a target cell ex vivo, then the target cell is re-introduced into an organism. In some such aspects, the target cell is cultured into multiple progeny cells ex vivo before being re-introduced in an organism. In more aspects, the organism is a human. In further aspects, the organism is a human patient. In certain aspects, the target cell was originally derived from the organism to which it is re-introduced. In other aspects, the target cell was originally derived from a different organism to which it is re-introduced.

In certain aspects, a Cpn60.1-related peptide used in the methods of the present invention and/or pharmaceutical compositions thereof are employed in combination therapies for treating or reducing the risk of a food allergy. In such aspects, administration can be in combination with one or more additional therapeutic agents. As used herein, the phrases “combination therapy,” “combined with,” “in combination,” and the like, refer to the use of more than one medication or treatment simultaneously to increase the response. In certain aspects, a Cpn60.1-related peptide used in the methods of the present invention and/or pharmaceutical compositions thereof are administered concurrently with, prior to, or subsequent to, one or more other desired therapeutics or medical procedures. In certain aspects, a Cpn60.1-related peptide used in the methods of the present invention and/or pharmaceutical compositions thereof are administered in combination together in a single composition or administered separately in different compositions.

In certain aspects, the particular combination of therapies to employ in a combination regimen will generally take into account the compatibility of the desired therapeutics and/or procedures, and the desired therapeutic effect to be achieved. In further aspects, the therapies employed may achieve a desired effect for the same purpose (e.g., a Cpn60.1-related peptide used in the methods of the present invention which is useful for treating, preventing, and/or delaying the onset of a food allergy may be administered concurrently with another therapeutic agent which is also useful for treating, preventing, and/or delaying the onset of a food allergy), or they may achieve different effects. In further aspects, the combination of therapies employed may achieve the same or a substantially similar desired effect for the same disease, condition or disorder; may achieve the same or a substantially similar desired effect for one or more different diseases, conditions or disorders; may achieve different desired effects for the same disease, condition or disorder; or may achieve different desired effects for one or more different diseases, conditions or disorders.

In additional aspects, the delivery of a Cpn60.1-related peptide used in the methods of the present invention as a pharmaceutical composition is in combination with one or more additional components that may improve the bioavailability of the Cpn60.1-related peptide used in the methods of the present invention, reduce and/or modify its metabolism, inhibit its excretion, and/or modify its distribution in the body.

In certain aspects, combination therapy may involve administrations of a plurality of Cpn60.1-related peptides in accordance with the present invention. In further aspects, combination therapy may involve administrations of a plurality of a Cpn60.1-related peptides that treat, prevent, improve, achieve remission of, and/or reduce the risk of a food allergy. In more aspects, combination therapy can be a plurality of Cpn60.1-related peptides that treat, prevent, improve, achieve remission of, and/or reduce the risk of multiple food allergies.

In certain aspects, a Cpn60.1-related peptide used in the methods of the present invention is combined with at least one pharmaceutically acceptable excipient, in the form of a pharmaceutical composition. As used herein, “pharmaceutical composition” refers to a formulation containing an active ingredient, and optionally a pharmaceutically acceptable carrier, diluent or excipient. The term “active ingredient” can interchangeably refer to an “effective ingredient,” and is meant to refer to any agent that is capable of inducing a sought-after effect upon administration. Examples of active ingredient include, but are not limited to, chemical compound, drug, therapeutic agent, small molecule, and the like.

In certain aspects of the present invention, the active ingredient is a Cpn60.1-related peptide as disclosed herein. In particular aspects, the active ingredient is PIN201104, or a derivative thereof. In certain aspects, the active ingredient is PIN201360, PIN201361, PIN201362, PIN201116, PIN201105 or a derivative thereof. In further aspects, the active ingredient is a peptide described in WO2009/106819, or a derivative thereof.

In certain aspects, the pharmaceutical compositions are useful in medicine or the manufacture of medicaments. In further aspects, the pharmaceutical compositions are useful in one or more of the therapeutic applications disclosed herein, for example, in an individual suffering from an autoimmune disorder. In additional aspects, the pharmaceutical compositions are formulated for administration to a human patient.

In certain aspects, the pharmaceutical composition is in a sterile injectable form (e.g., a form that is suitable for subcutaneous injection or intravenous infusion). In more aspects, the pharmaceutical composition is in a liquid dosage form that is suitable for injection. In further aspects, the pharmaceutical composition is in a powder (e.g., lyophilized and/or sterilized), optionally under vacuum, which is reconstituted with an aqueous diluent (e.g., water; buffer; salt solution, and the like) prior to injection. In additional aspects, the pharmaceutical composition is diluted and/or reconstituted in an aqueous diluent (e.g., water, sodium chloride solution, sodium acetate solution, benzyl alcohol solution, phosphate buffered saline, and the like). In certain aspects, the pharmaceutical composition is in a form that can be refrigerated and/or frozen. In further aspects, the pharmaceutical composition is in a form that cannot be refrigerated and/or frozen. In certain aspects, the pharmaceutical composition is a reconstituted solution and/or liquid dosage form which can be stored for a certain period of time after reconstitution (e.g., 2 hours, 12 hours, 24 hours, 2 days, 5 days, 7 days, 10 days, 2 weeks, a month, two months, or longer).

In certain aspects, preparatory methods for pharmaceutical compositions include the step of bringing the active ingredient (e.g., a Cpn60.1-related peptide used in the methods of the present invention) into association with one or more pharmaceutically acceptable excipients and then shaping and/or packaging the product into a desired single- or multi-dose unit. A pharmaceutical composition in accordance with the invention may be prepared, packaged in bulk, packaged as a single unit dose, and/or packaged as a plurality of single unit doses. As used herein, a “unit dose” refers to a discrete amount of the pharmaceutical composition including a predetermined amount of the active ingredient. The amount of the active ingredient is generally equal to a dose that would be administered to a subject and/or a convenient fraction of such a dose such as, for example, one-half or one-third of such a dose. The relative amounts of active ingredient, pharmaceutically acceptable excipient, and/or any additional ingredients in a pharmaceutical composition in accordance with the invention may vary, depending upon the identity, size, and/or condition of the subject treated and/or depending upon the route by which the composition is to be administered. In certain aspects, for example, the composition may include between about 0.1% to 100% (w/w) of an active ingredient.

In another aspect, the present invention includes kits that are useful for carrying out the methods of the present invention. The components contained in the kit depend on a number of factors, including the particular application (e.g., the particular route of administration to be employed, or the particular disease, condition or disorder to be treated). In certain aspects, the present invention provides a kit for administering a Cpn60.1-related peptide in accordance with the present invention to treat a disease, condition or disorder disclosed herein. In some such aspects, the kit further includes instructions for administration. In certain aspects, the kit is for administering a Cpn60.1-related peptide to treat a patient with a food allergy. In certain aspects, the kits contain one or more a Cpn60.1-related peptides. In certain aspects, the kit includes a number of unit doses of a pharmaceutical composition containing a Cpn60.1-related peptide. In additional aspects, kits for use in accordance with the present invention include instructions (e.g., for administration, for storage, and the like), buffers and/or other reagents. In some such aspects, the kit includes (i) at least one Cpn60.1-related peptide, (ii) a syringe, needle, applicator, or the like for administration of the at least one Cpn60.1-related peptide to a patient, and (iii) instructions for use. In further aspects, the kit includes a treatment schedule designating when the unit dosages are to be administered. In more aspects, placebo dosages, either in a form similar to or distinct from the dosages of the pharmaceutical compositions, are included. In certain aspects, kits include one or more containers so that certain of the individual components or reagents may be separately housed. In certain aspects, kits may include a means for enclosing the individual containers in relatively close confinement for commercial sale, e.g., a plastic box, in which instructions, packaging materials such as Styrofoam, and the like, may be enclosed.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Example 1 Effectiveness of CPN60.1-Related Peptides in an Animal Model of Ovalbumin-Induced Food Allergy

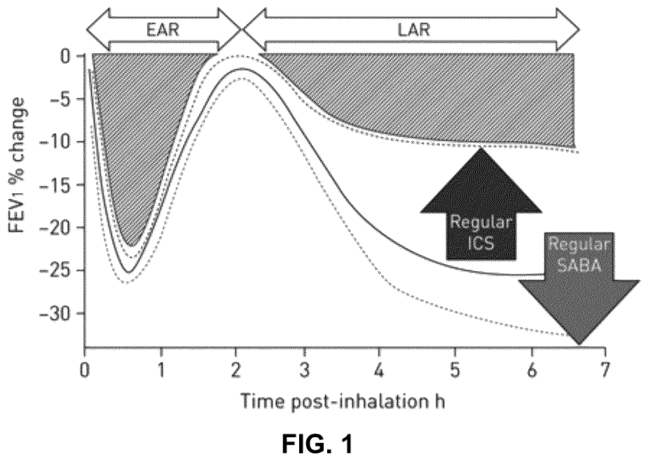

Chaperonin 60.1 (Cpn60.1)-related peptides, such as PIN201104 (“'1104”), have been shown to be very effective in blocking the inflammation associated with the late phase response. See, FIG. 1. However, previous work did not look at the effect of Cpn60.1-related peptides on the early phase response, which is characterized by an acute anaphylaxis where mast cells and basophils play an important role. For some indications, such as food allergies, therapeutic effectiveness in the early phase is critical. The present study assessed whether 1104 has an effect on the early response in addition to its known effects on the late response. A mouse model of food allergy was developed that showed an ovalbumin (OVA)-induced progressive severity of anaphylaxis.

Allergens, whether from food, air pollution, dust mites, pet dander, or mold, can induce hypersensitivity reactions mediated by IgE antibodies. An outcome of an allergic reaction is an asthmatic response characterized by airflow obstruction and bronchospasm. In allergic responses, T-Helper cells induce expression of many cytokines including TNF-α, IL-2, IL-4, IL-5, IL-6, IL-10, and IL-13 (1-4). Of these cytokines, IL-4 and IL-13 play critical roles in the allergic response. IL-13 has a distinctive role in mucus production, while IL-4 has been found to be the major driver of IgE and IgG1 synthesis by B-Cells. Furthermore, IL-5 drives blood and tissue eosinophilia, a characteristic feature of allergic reactions.^{25,26}

Ovalbumin (OVA) and house dust mite (HDM) have been widely used antigens for inducing allergic responses.^{27,28,29,30} for example, both the OVA-Induced Asthma Model and the HDM-Induced Asthma Model can elicit airway inflammation, which can lead to airflow obstruction and airway remodeling (AR).

There are many chemokines associated with allergic reactions, including CCL11, CCL17 and CCL22, which have all been found upregulated in the broncho-alveolar lavage of patients with asthma. In a murine model, it has been shown that the suppression of CCL11 decreased eosinophil recruitment and reduced airway hyper-responsiveness. Indeed, asthmatic patient samples displayed higher levels of CCL11 in plasma than healthy volunteers. Chemokines CCL17 and CCL22 bind to CCR4 which is expressed mostly by Th2 cells and play a major role in the recruitment of T cells both in mice and in humans. CCL17 and CCL22 are well documented in many allergic reactions including skin and food allergies. In addition, IL-4, IL-5, IL-13 and INF-γ, which are Th1/Th2 cytokines, have been reported to be upregulated.³¹

### Mouse Model of Food Allergy

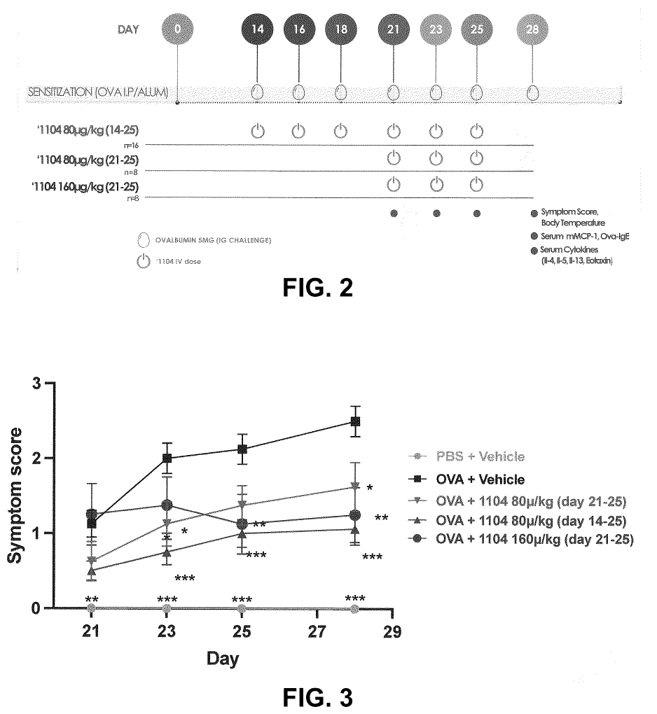

In the present study, a mouse model of food allergy was developed that showed an ovalbumin (OVA)-induced progressive severity of anaphylaxis. The design protocol and endpoints are illustrated in FIG. 2 and summarized in Table 1.

TABLE 1
Food Allergy Model

| | | Challenge i.p. | Challenge i.g. 200 μL every | |
|---|---|---|---|---|
| Group | N | on day 0 | other day, days 14-28 | Drug treatment i.v. |
| 1 | 8 | Saline | Saline | Vehicle |
| 2 | 8 | OVA (100 μg) in | OVA 5 mg | Vehicle |
| 3 | 8 | Alum (1 mg) | | ′1104: 80 μg/kg; 6 doses on |
| | | | | days 14, 16, 18, 21, 23, and 25 |
| 4 | 8 | | | ′1104: 80 μg/kg; 3 doses on |
| | | | | days 21, 23, and 25 |
| 5 | 8 | | | ′1104: 160 μg/kg; 3 doses on |
| | | | | days 21, 23, and 25 |

Endpoints:
1. Anaphylaxis scoring (days 14-28)
2. Body temperature (days 14-28)
3. Terminal Blood Serum - biomarkers and OVA-specific IgE assay
4. Tissue collection (histopathology)

The clinical scale used for the anaphylaxis scoring is provided in Table 2.

TABLE 2
Clinical Scoring Scale*

| Score | Scale |
|---|---|
| 0 | No symptoms |
| 1 | Scratching and rubbing around the nose and head |
| 2 | Puffiness around the eyes and mouth, diarrhea, |
| | pilar erection, reduced activity, |
| | and/or decreased activity with increased respiratory rate |
| 3 | Wheezing, labored respiration, and cyanosis around |
| | the mouth and the tail |
| 4 | No activity after prodding or tremor and convulsion |
| 5 | Death |

*This Clinical Scoring Scale is a validated anaphylaxis scale commonly used in murine food allergy studies to assess symptom severity.^{32, 33}

Animals: Male BalbC mice (20-30 g on arrival, Charles Rivers, UK) were used in the present study.

Animal welfare: On arrival from the supplier, animals were acclimatized for period of 7 days before start of experimental procedures. Mice were housed in cages of four on arrival based on weight (equal distribution of animal weights amongst each of the cages by the animal technician) with a 12 hour light dark cycle. Room temperature and humidity were maintained between 17-24° C. and 40-70%, respectively. Environmental enrichment was provided in all cages. Mice had access to standard chow ad libitum and Water was available from bottles adlibitum.

Animals were placed into cages of four, as outlined below, and the welfare of all animals were checked on a daily basis. Throughout the study, the following guidelines were used to assess non-specific or unexpected adverse effects in animals undergoing regulated, relating to either the procedure or test compound dosing.
Body weight loss greater than 20% of the highest measured individual body weight.
Marked piloerection with other signs of dehydration such as skin tenting.
Unresponsive to activity and provocation.
Hunched persistently (frozen).
Distressed-persistent vocalization.
Oculo-nasal discharge persistent and copious.
Labored respiration.
Persistent tremors.
Persistent convulsions.

Animals showing two or more of any of the limiting clinical signs in the category equivalent to the protocol severity limit were removed from the study and were killed by a schedule 1 method (cervical dislocation). Where an animal reached the limit of either or both of the first two signs with or without any other signs, it was removed from the study and killed by a schedule 1 method at the establishment. However, no animals were removed during this study.

OVA sensitization and challenge: Mice were sensitized intraperitoneally with 25 μL on day 0 with 100 μg of ovalbumin adsorbed to 1 mg of alum (Imject Alum—Aluminum content 40 mg/mL). Mice were then challenged via oral gavage with 5 mg of OVA in 200 μL or with 200 μL of PBS on days 14, 16, 18, 21, 23, 25, and 28 (Groups 1-5).

Body temperature and anaphylaxis assessment: After each challenge, animals were monitored for 60 min, and rectal temperatures were recorded at 0, 15, 30, and 60 min following oral gavage with PBS or OVA. A score of 0-5 were assigned to each mouse based on anaphylaxis symptoms. The clinical scale used for the anaphylaxis scoring is provided in Table 2 above.

Treatment of animals with '1104 or vehicle: Mice were weighed and placed into a hotbox for 5 min before being placed in a whole-body restrainer to receive an intravenous administration (5 mL/kg) via the tail vein of either '1104 (80 μg/kg or 800 μg/kg) or vehicle 15 min before OVA challenge on days 14, 16, 18, 21, 23 and 25.

Blood sample collection: One hour after the final OVA challenge on day 28 or day 38, a terminal blood sample was collected via cardiac puncture and placed into a serum tube. Each serum sample was kept at room temperature for 45 minutes to allow coagulation, before being centrifuged (2000×g, 15 min at 4° C.) from which the resulting supernatant was extracted, aliquoted and stored at −80° C. for analysis.

Tissue collection: Immediately after collecting the terminal blood samples, the animals were culled by an overdose with pentobarbital. The abdomen was opened up and the small intestine dissected free from each animal and flushed with PBS. The tissue were then divided into duodenum, jejunum, and ileum. Each section was opened longitudinally and coiled onto a wooden stick to create a roll. Each tissue roll was then sectioned, and one section was placed in 10% formalin for 48 h before being transferred into 70% ethanol for future histopathology. The second section was placed into a sterile Eppendorf, snap frozen and then stored at −80° C. for future biomarker analysis.

Following collection of the small intestine, the mesenteric lymph nodes were dissected free from each animal. Following dissection, the lymph node were placed into a sterile Eppendorf, snap frozen and then stored at −80° C. for future analysis.

OVA specific IgE and mMCP1 ELISA assay: Serum supernatant was evaluated for OVA specific IgE and mMCP1 concentrations using ELISA kit (AssayGenie and Invitrogen respectively) as per the manufacturer's instructions. Optical density was measured at 450 nM using a microplate reader (SpectraMax 340PC). Concentrations of IgE were determined using SoftMax Pro v.6.4 (Molecular Devices). Data were reported as OVA specific IgE (μg/mL) or mMCP1 concentrations (μg/mL), mean±S.E.M. (standard error of the mean).

Cytokine/Chemokine assays: Cytokine/chemokine concentrations (see below for details of biomarkers to be evaluated) of serum samples (all groups) were measured using magnetic multiplex assays as per the manufacturer's instructions. Levels were measured using a Magpix system (Luminex Corp.).

Optical density was measured at 450 nM using a microplate reader (SpectraMax 340PC). Mouse cytokine/chemokine magnetic multiplex panel Biotechne): IL-4, IL-5 IL-13, and Eotaxin. Data were reported as cytokine/chemokine (μg/mL), mean±S.E.M. (standard error of the mean).

Data Analysis: Inter-group deviations were statistically analyzed by a one-way analysis of variance (ANOVA). In the case of significant difference in the mean values among the different levels of treatment, comparisons versus the vehicle group were carried out using the Dunnett's test. p<0.05 was considered statistically significant.

### Results

Clinical Scoring: The present model provides a progressive model of food allergy. As shown in FIG. 3, an increase in responses was observed over time in all OVA exposed mice. Clinical scoring of animals was conducted at days 21, 23, 25, and 28. As [symbol] expected, mice challenged with 5 mg OVA (i.g.) every other day from days 14-28 ( [symbol] ) displayed a statistically significant progressively increased severity of anaphylaxis over PBS treated animals ( [symbol] ). By day 28, some animals in the OVA/vehicle arm scored a 4 on the anaphylaxis symptom scale. Treatment of animals with three doses ( [symbol] ) of '1104 (80 g/kg) significantly reduced the severity of anaphylaxis over vehicle group exposed to OVA ( [symbol] ) at day 28, whereas treatment six doses ( [symbol] ) of '1104 (80 g/kg) significantly reduced the severity of anaphylaxis over vehicle group exposed to OVA ( [symbol] ) at days 23, 25, and 28. Treatment with three higher dose of '1104 (160 g/kg) ( [symbol] ) significantly reduced the severity of anaphylaxis over vehicle group exposed to OVA ( [symbol] ) at days 25 and 28, similar to the group receiving six doses of the lower 80 g/kg. Interestingly, the final day results confirmed that Cpn60.1-related peptides have short pharmacokinetics (PK) and long pharmacodynamics (PD) hypothesis, as serum PK of '1104 is approximately 15 minutes while the last OVA challenge was 3 days after the last '1104 dose.

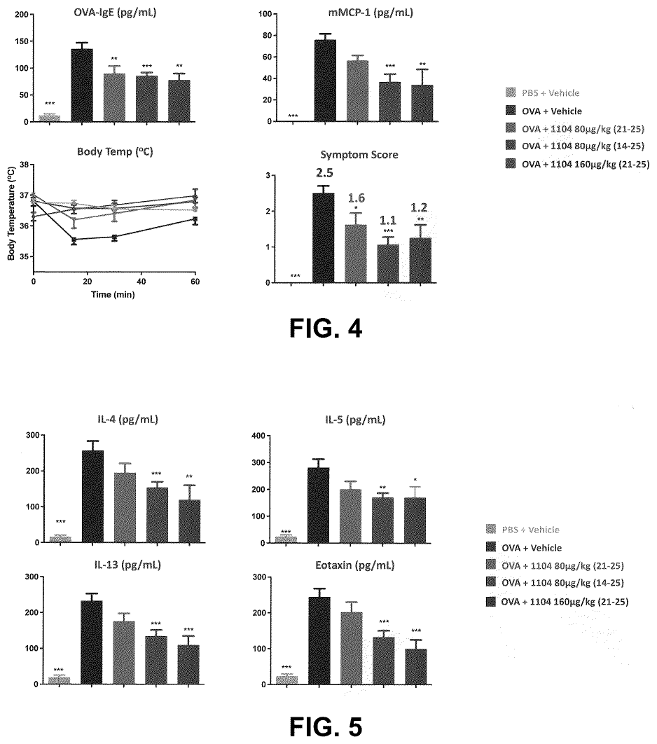

Biometrics: The biomarker analysis was performed on day 28, where the most pronounced difference between the vehicle and '1104 treatment groups were observed. OVA-specific IgE, mMCP, body temperature, and clinical scores were recorded. As shown in FIG. 4, prophylactic and therapeutic dosing with '1104 (80 or 160 g/kg; three or six doses) significantly reduced all four biometrics of food allergy at day 28, three days after the last '1104 dose: OVA-specific immunoglobulin E (IgE) (upper left panel); murine mast cell protease (mMCP-1) (upper right panel); body temperature (bottom left panel); and clinical scoring (bottom right panel).

Key Th2 Cytokines/Chemokines: The serum levels of key Th2 cytokines/chemokines were also measured on day 28: IL-4, IL-5, IL-13, and Eotaxin. As expected, mice challenged with 5 mg OVA (i.g.) every other day from days 14-28 displayed a statistically increased levels of these key Th2 cytokines/chemokines when compared to PBS treated animals. See, FIG. 5: IL-4 (upper left panel); IL-5 (upper right panel); IL-13 (bottom left panel); and Eotaxin (bottom right panel). Confirming the biometrics results described above, six doses of 80 g/kg and three doses of 160 g/kg '1104 significantly reduced levels of the key Th2 cytokines/chemokines in ovalbumin-induced food allergy mouse model, as would be expected with the '1104 reduction of the severity of anaphylaxis in these animals.

### Conclusion

The results of present study using a mouse model of food allergy suggests that Cpn60.1-related peptides are effective for the prevention and treatment of food allergies, representing a novel non-allergen-specific therapy for food allergies. This study provides the first evidence that Cpn60.1-related peptides have an effect on the early response in addition to its previously known effects on the early response.

### Example 2 Long-Lasting Effectiveness of CPN60.1-Related Peptides in an Animal Model of Ovalbumin-Induced Food Allergy

As shown in Example 1, Cpn60.1-related peptides were shown to be effective for the prevention and treatment of food allergies. The present study assessed the effectiveness of a higher dose of '1104 (800 g/kg) and the long-lasting effectiveness of '1104 in preventing of food allergies after a longer period after administration of '1104 in the animal model of ovalbumin (OVA)-induced of food allergy described above.

### Materials and Methods

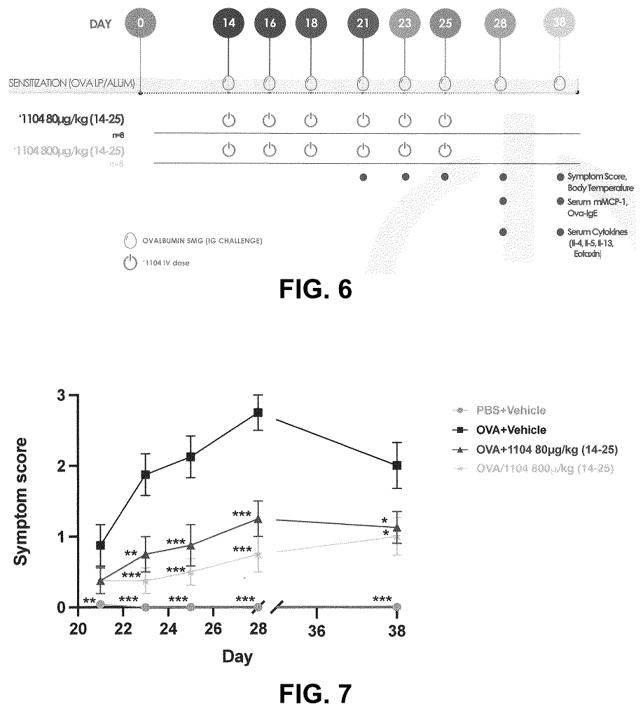

Study Design: The design protocol and endpoints are illustrated in FIG. 6 and summarized in Table 3.

TABLE 3
Study Design

| | | Challenge i.g. | Drug treatment (5 mL/kg, | Measurements and | Measurements and |
|---|---|---|---|---|---|
| | Challenge i.p. | (200 μL dose volume, | i.v., a) treatment days 14, | End Point sample | End Point sample |
| Group | (day 0) | q.o.d., day 14-28) | 16, 18, 21, 23 and 25 | collection (day 28) | collection (day 38) |
| 1 | Saline | Saline | Vehicle | Blood Serum | |
| | | | | Tissue collection | |
| 2 | OVA | Saline | Vehicle | Blood Serum | |
| | | | | Tissue collection | |
| 3 | OVA | OVA | Vehicle | Blood Serum | |
| | | | | Tissue collection | |
| 4 | OVA | OVA | ′1104: 80 μg/kg | Blood Serum | |
| | | | | Tissue collection | |
| 5 | OVA | OVA | ′1104: 800 μg/kg | Blood Serum | |
| | | | | Tissue collection | |
| 6 | Saline | Saline | Vehicle | | Blood Serum |
| | | | | | Tissue collection |
| 7 | OVA | Saline | Vehicle | | Blood Serum |
| | | | | | Tissue collection |
| 8 | OVA | OVA | Vehicle | | Blood Serum |
| | | | | | Tissue collection |
| 9 | OVA | OVA | ′1104: 80 μg/kg | | Blood Serum |
| | | | | | Tissue collection |
| 10 | OVA | OVA | ′1104: 800 μg/kg | | Blood Serum |
| | | | | | Tissue collection |

N = 8 for each group.

Animals: As described in Example 1.

Animal welfare: As described in Example 1.

OVA sensitization and challenge: For animals from groups 1-5, ss described in Example 1. Animals from groups 6-10 were left to rest for 10 days and then received an extra challenge at day 38.

Body temperature and anaphylaxis assessment: As described in Example 1.

Treatment of animals with '1104 or vehicle: Mice were weighed and placed into a hotbox for 5 min before being placed in a whole-body restrainer to receive an intravenous administration (5 mL/kg) via the tail vein of either '1104 (80 μg/kg or 800 μg/kg) or vehicle 15 min before OVA challenge on days 14, 16, 18, 21, 23 and 25.

Blood sample collection: One hour after the final OVA challenge on day 28 or day 38, a terminal blood sample was collected via cardiac puncture and placed into a serum tube. Each serum sample was kept at room temperature for 45 minutes to allow coagulation, before being centrifuged (2000×g, 15 min at 4° C.) from which the resulting supernatant was extracted, aliquoted and stored at −80° C. for analysis.

Tissue collection: As described in Example 1.

OVA specific IgE and mMCP1 ELISA assay: As described in Example 1.

Cytokine/Chemokine assays: Cytokine/chemokine concentrations (see below for details of biomarkers to be evaluated) of serum samples (all groups) were measured using magnetic multiplex assays as per the manufacturer's instructions. Levels were measured using a Magpix system (Luminex Corp.).

IL-9 and CCL-17 were measured using commercial ELISA kit (Biotechne, UK) as per the manufacturer's instructions. Optical density was measured at 450 nM using a microplate reader (SpectraMax 340PC). Concentrations of IL-9 and CCL-17 will be determined using SoftMax Pro v. 6.4 (Molecular Devices). Mouse cytokine/chemokine magnetic multiplex panel Biotechne): IL-4, IL-5, IL-10, IL-13, IL-17A, Eotaxin, CCL-22, IFN-gamma, TNF alpha. Data were reported as cytokine/chemokine (μg/mL), mean±S.E.M. (standard error of the mean).

Data Analysis: As described in Example 1.

### Results

Clinical Scoring: The present model provides a progressive model of food allergy. As shown in FIG. 7, an increase in responses was observed over time in all mice exposed to OVA. Clinical scoring of animals was conducted at daiys 21, 23, 25, and 28. As expected, treatment of animals with six doses ( [symbol] ) of '1104 (80 g/kg), significantly reduced the severity of anaphylaxis over vehicle group exposed to OVA ( [symbol] ) at days 23, 25, and 28. Treatment with a higher dose of '1104 (800 g/kg) ( [symbol] ) significantly reduced the severity of anaphylaxis over vehicle group exposed to OVA ( [symbol] ) at days 23, 25, and 28. Interestingly, both doses of '1104 (80 and 800 g/kg) displayed long-lasting effectiveness by significantly decreasing the severity of anaphylaxis over vehicle group exposed to OVA 13 days after the last '1104 dose. These results again confirmed that Cpn60.1-related peptides have short pharmacokinetics (PK) and long pharmacodynamics (PD).

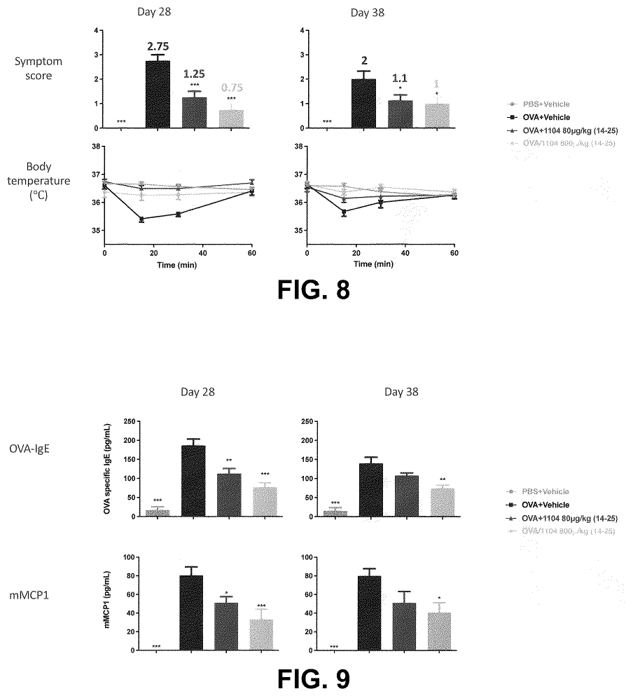

Biometrics: The biomarker analysis was performed on day 28 and again on day 38. Consistent with the results in Example 1, '1104 (80 and 800 g/kg; six doses) significantly improved symptom scores and body temperature over vehicle group exposed to OVA at day 28 (FIG. 8, left panels). Interestingly, these beneficial effects were maintained at day 38, without further dosing with '1104 (FIG. 8, right panels).

OVA specific IgE and mMCP1 levels at Day 28 and at Day 38: In one group of mice, the biomarker analysis was performed on serum samples obtained on day 28 from OVA sensitized mice (day 0) that were challenged via oral gavage with OVA on days 14, 16, 18, 21, 23 and 25 with vehicle or '1104 (80 or 800 μg/kg, i.v.). Samples were collected 1 h after the final OVA challenge on day 28. Both doses of '1104 significantly reduced the OVA-specific IgE levels (FIG. 9, top left panel) and mMCP-1 levels (FIG. 9, bottom left panel) clinical scores at day 28, three days after the last '1104 dose. These data confirmed the results of Example 1.

In another group of mice, the biomarker analysis was performed on serum samples obtained on day 38 from OVA sensitized mice (day 0) that were challenged via oral gavage with OVA on days 14, 16, 18, 21, 23 and 25 with vehicle or '1104 (80 or 800 μg/kg, i.v.). Animals from the groups sampled on day 38 also received an additional OVA challenge on day 38. Samples were collected 1 h after the final OVA challenge on 38. Again, both doses of '1104 reduced the OVA-specific IgE (FIG. 9, top right panel) and mMCP-1 levels (FIG. 9, bottom right panel), although only the higher dose reached statistical significance. Interesting, these data indicate that, despite a very short half-life (˜10-15 min across species), '1104 exhibits a long-lasting effect in this model of food allergy, thus supporting a prophylactic role as well as a therapeutic role for food allergies.

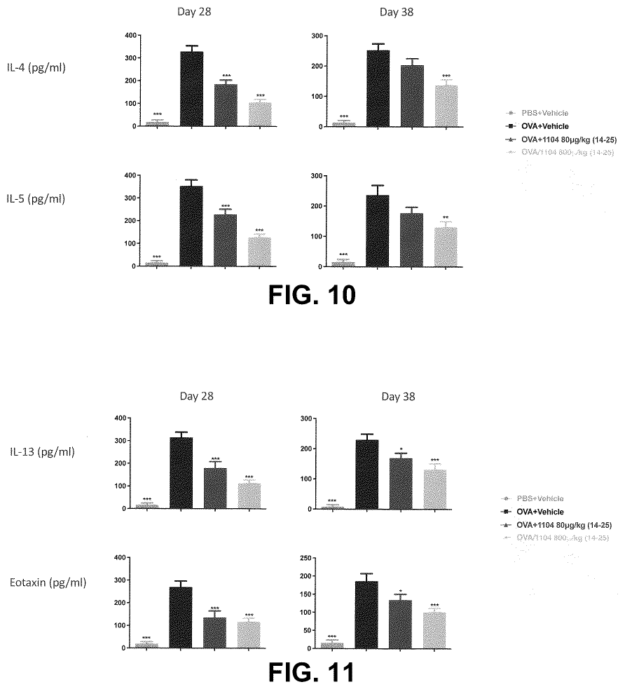

Key Cytokines/Chemokines: The serum levels of key Th2 cytokines/chemokines were measured on day 28 and day 38. As expected, mice challenged with 5 mg OVA (i.g.) every other day from days 14-28 displayed a statistically increased levels of these key cytokines/chemokines when compared to PBS treated animals. See, FIGS. 10-14.

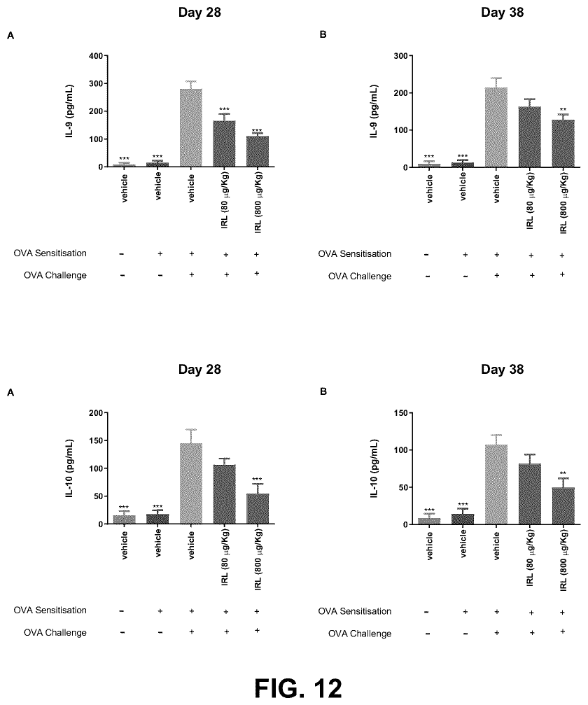

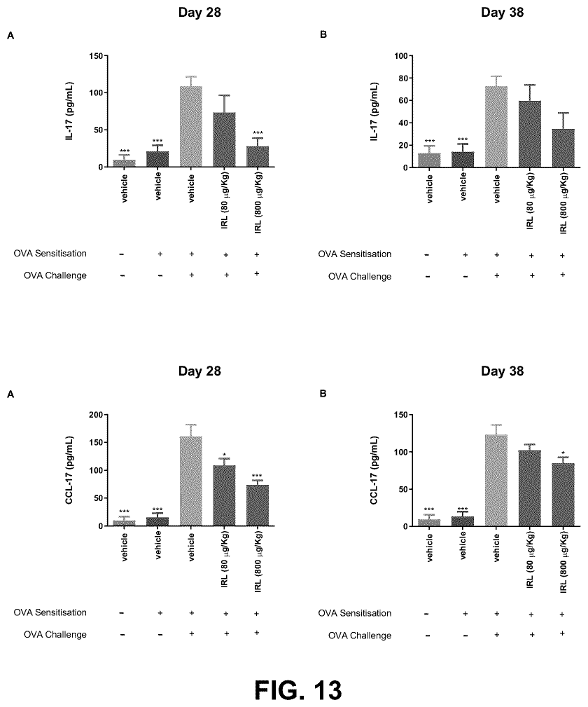

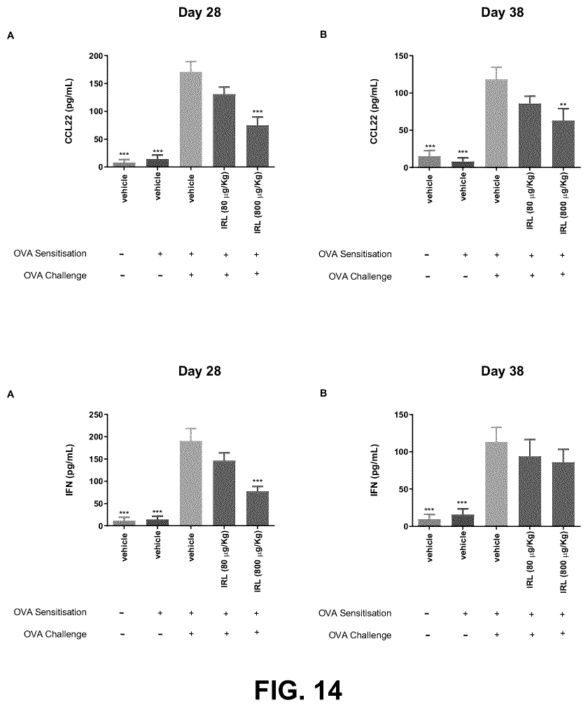

Confirming the biometrics results described above, six doses of 80 g/kg and 800 g/kg '1104 significantly reduced levels of the key cytokines/chemokines in ovalbumin-induced food allergy mouse model, as would be expected with the '1104 reduction of the severity of anaphylaxis in these animals. The key cytokines/chemokines included IL-4 (FIG. 10, upper panels); IL-5 (FIG. 10, lower panels); IL-9 (FIG. 12, upper panels); IL-10 (FIG. 12, lower panels); IL-13 (FIG. 11, upper panels); IL-17 (FIG. 13, upper panels); CCL-17 (FIG. 13, lower panels); CCL-22 (FIG. 14, upper panels); IFN (FIG. 14, lower panels); and Eotaxin (FIG. 11, lower panels). Significant reductions in the levels of the key Th2 cytokines/chemokines were observed at day 28, three days after the last dose of '1104 (left panels) and at day 28, 13 days after the last dose of '1104 (right panels).

Interestingly, IL-9 has recently been reported to have a significant role in the development of IgE-mediated food allergy.³⁴ For example, IL-9 has been reported to be a key cytokine promoting mast cell expansion and has been shown to be derived from peTh2 cells in food allergy.³⁵ As such, it is being viewed as a potential target for food allergy treatment. The above results showing the significant reduction of IL-9 (FIG. 12, upper panels) induced by '1104 in the animal model of food allergy supports the use of Cpn60.1-related peptides for the prevention and treatment of food allergies.

### Conclusion

The results of present studies using a mouse model of food allergy suggests that Cpn60.1-related peptides are effective for the prevention and treatment of food allergies. Cpn60.1-related peptides attenuated the early anaphylactic allergic response in a model of ovalbumin driven food allergy. The effect, after both prophylactic and therapeutic dosing, was consistent across “clinical” symptoms (anaphylaxis score and body temperature) and relevant serum biomarkers (i.e., mMCP-1, OVA-IgE, IL-4, IL-5, IL-13, and Eotaxin). Despite a short plasma half-life (˜15 min), Cpn60.1-related peptides showed, in this early allergic response model, a long pharmacodynamic effect (13 days) consistent with the effect observed in models of late allergic response. Accordingly, these studies support the use of Cpn60.1-related peptides as an alternative non-allergen specific therapy for the treatment of food allergy.

### REFERENCES

¹ Prescott, S., et al. (2011) “Food allergy: Riding the second wave of the allergy epidemic.” Pediatr. Allergy Immunol. 22: 155-160.
² Osborne, N. J., et al. (2011) “Prevalence of challenge-proven IgE-mediated food allergy using population-based sampling and predetermined challenge criteria in infants.” J. Allergy Clin. Immunol. 127: 668-676.
³ Leung, A. S. Y., et al. (2018). “Food allergy in the developing world.” J. Allergy Clin. Immunol. 141: 76-78.
⁴ Gupta, R. S., et al. (2019). “Prevalence and Severity of Food Allergies Among US Adults.” JAMA Netw Open. 2(1): e185630.
⁵ United States Census Bureau Quick Facts (2015 and 2016 estimates).
⁶ Reuters Health (2010). “Rates of food sensitivity vary by country: study.” Health & Pharma, Mar. 12, 2010.
⁷ Isselbacher, et al. (1996). HARRISON'S PRINCIPLES OF INTERNAL MEDICINE, 13 ed., 1814-1882.
⁸ THE MERCK MANUAL OF DIAGNOSIS AND THERAPY, (2011). 19^{th} Edition, published by Merck Sharp & Dohme Corp., (ISBN 978-O-911910-19-3).
⁹ THE ENCYCLOPEDIA OF MOLECULAR CELL BIOLOGY AND MOLECULAR MEDICINE, Robert S. Porter et al. (eds.), published by Blackwell Science Ltd., 1999-2012 (ISBN 9783527600908).
10 MOLECULAR BIOLOGY AND BIOTECHNOLOGY: A COMPREHENSIVE DESK REFERENCE, (1995). Robert A. Meyers (ed.), published by VCH Publishers, Inc. (ISBN 1-56081-569-8).
¹¹ IMMUNOLOGY, (2006). Werner Luttmann, published by Elsevier.
¹² JANEWAY'S IMMUNOBIOLOGY, (2014). Kenneth Murphy, Allan Mowat, Casey Weaver (eds.), Taylor & Francis Limited, (ISBN 0815345305, 9780815345305).
¹³ LEWIN'S GENES XI, (2014). published by Jones & Bartlett Publishers (ISBN-1449659055).
¹⁴ Michael Richard Green and Joseph Sambrook, (2012). MOLECULAR CLONING: A LABORATORY MANUAL, 4th ed., Cold Spring Harbor Laboratory Press, Cold Spring Harbor, N.Y., USA (ISBN 1936113414).
¹⁵ Davis et al., (2012). BASIC METHODS IN MOLECULAR BIOLOGY, Elsevier Science Publishing, Inc., New York, USA (ISBN 044460149X).
¹⁶ LABORATORY METHODS IN ENZYMOLOGY: DNA, (2013). Jon Lorsch (ed.) Elsevier (ISBN 0124199542).
¹⁷ CURRENT PROTOCOLS IN MOLECULAR BIOLOGY (CPMB), (2014). Frederick M. Ausubel (ed.), John Wiley and Sons (ISBN 047150338X, 9780471503385).
¹⁸ CURRENT PROTOCOLS IN PROTEIN SCIENCE (CPPS), (2005). John E. Coligan (ed.), John Wiley and Sons, Inc.
¹⁹ CURRENT PROTOCOLS IN IMMUNOLOGY (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
²⁰ Gould, H.J., et al. (2003). “The biology of IGE and the basis of allergic disease.” Annual Review of Immunology 21: 579-628.
²¹ United States Patent Published Patent Application No. 20040132163 entitled: “Biological materials and uses thereof.” Published: Jul. 8, 2004.
²² U.S. Pat. No. 11,098,090 entitled: “Mycobacteria tuberculosis chaperonin 60.1 peptides and uses thereof.” Issued: Aug. 24, 2021.
²³ U.S. Pat. No. 9,320,791 entitled: “Peptides from the polypeptide chaperonin 60.1, and their use in medicine for the treatment of inflammatory conditions are described.” Issued: Apr. 26, 2016.
²⁴ U.S. Pat. No. 9,085,632 entitled: “Biological materials and uses thereof.” Issued: Jul. 21, 2015.
²⁵ Lambrecht, B., et al. (2019). “The Cytokines of Asthma.” Immunity 50: 975-991.
²⁶ Castan, L., et al. (2017). “Chemokine receptors in allergic diseases.” Allergy 72: 682-690.
²⁷ Nials, A. and Uddin, S. (2008). “Mouse models of allergic asthma: acute and chronic allergen challenge. Dis. Model Mech. 1, 213-220.
²⁸ Gandhi, V., et al. (2013). “House dust mite interactions with airway epithelium: role in allergic airway inflammation.” Curr. Allergy Asthma Rep. 13: 262-270.
²⁹ Castan, L., et al. (2017). “Chemokine receptors in allergic diseases.” Allergy 72: 682-690.
³⁰ Kim, J., et al. (2018). “FcγR/ROS/CK2α Is the Key Inducer of NF-κB Activation in a Murine Model of Asthma.” Int. Arch. Allergy Immunol. 175: 16-25.
³¹ Qian, G., et al. (2018). “LPS inactivation by a host lipase allows lung epithelial cell sensitization for allergic asthma.” J. Exp. Med. 215: 2397-2412.
³² Li, X-M, et al. (2000). “A murine model of peanut anaphylaxis: T- and B-cell responses to a major peanut allergen mimic human response.” J. Allergy Clin. Immunol. 106: 150-158.
³³ Schülke S. and Albrecht, M. (2019). “Mouse Models for Food Allergies: Where Do We Stand?” Cells 8(6): 546.
³⁴ See, e.g., Shik, D., et al. (2017). “IL-9-producing cells in the development of IgE-mediated food allergy.” Semin Immunopathol., 39(1): 69-77.
Lee, J.B. (2016). “Regulation of IgE-Mediated Food Allergy by IL-9 Producing Mucosal Mast Cells and Type 2 Innate Lymphoid Cells.” Immune Netw. 16(4): 211-218.
Chen, C.-Y., et al. (2015). “Induction of Interleukin-9-Producing Mucosal Mast Cells Promotes Susceptibility to IgE-Mediated Experimental Food Allergy.” Immunity 43, 788-802.
Xie, J., et al. (2012). “Elevated Antigen-Driven IL-9 Responses Are Prominent in Peanut Allergic Humans.” PLoS ONE 7(10): e45377.
³⁵ Berin, M.C. (2023). “Targeting type 2 immunity and the future of food allergy treatment.” J. Exp. Med., 220(4): e20221104.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain, using no more than routine experimentation, many equivalents to the specific embodiments described herein. Such equivalents are intended to be encompassed by the following claims.

SEQUENCES

DGSVVVNKVSELPAGHGLNVNTLSYGDLAAD (SEQ ID NO: 1;
'1104);
DGSVVVNKVSELPAGH (SEQ ID NO: 2);
GLNVNTLSYGDLAAD (SEQ ID NO: 3);
SELPAGHGLNVNLTS (SEQ ID NO: 4);
DGSVVVNKVS (SEQ ID NO: 5);
ELPAGHGLNV (SEQ ID NO: 6);
NTLSYGDLAAD (SEQ ID NO: 7)

1. A method for treating or preventing the onset of a food allergy or food intolerance to a dietary antigen derived from a food in a subject in need thereof, the method comprising administering to the subject, a peptide related to Chaperonin 60.1 (Cpn60.1).

2. The method of claim 1, wherein the Cpn60.1-related peptide is selected from the group consisting of:
(SEQ ID NO: 1; 1104)
DGSVVVNKVSELPAGHGLNVNTLSYGDLAAD;
(SEQ ID NO: 2)
DGSVVVNKVSELPAGH;
(SEQ ID NO: 3)
GLNVNTLSYGDLAAD;
(SEQ ID NO: 4)
SELPAGHGLNVNLTS;
(SEQ ID NO: 5)
DGSVVVNKVS;
(SEQ ID NO: 6)
ELPAGHGLNV;
(SEQ ID NO: 7)
NTLSYGDLAAD;
and
or a functionally equivalent fragment or variant thereof.

3. The method of claim 2, wherein the Cpn60.1-related peptide is SEQ ID NO:1.

4. The method of any one of claims 1-3, further comprising, prior to administration, diagnosing the subject as having, or likely to develop, a food allergy or food intolerance, or receiving the results of an assay that diagnoses the subject as having, or likely to develop, a food allergy or food intolerance.

5. The method of any one of claims 1-4, wherein the Cpn60.1-related peptide is administered prior to the first exposure to a potential causal food allergen, or upon clinical signs of atopic symptoms.

6. The method of any one of claims 1-5, wherein the subject has been diagnosed with at least one food allergy or food intolerance.

7. The method of any one of claims 1-6, wherein the dietary antigen is derived from milk and products thereof; eggs and products thereof; meat and products thereof; fish, mollusks, and crustaceans and products thereof; oils, fats, and products thereof; grains and products thereof; pulses, seeds, kernels, nuts, and products thereof; vegetables and products thereof; fruits and products thereof; mushrooms and products thereof; sugar, sugar products, chocolate products, and confectionary; and spices and herbs.

8. The method of any one of claims 1-7, wherein the administration of the Cpn60.1-related peptide is acute or short-term.

9. The method of any one of claims 1-7, wherein the administration of the Cpn60.1-related peptide is long-term.

10. A therapeutically effective amount of a peptide related to Cpn60.1 for use in a method for preventing the onset of, or for treating a food allergy or food intolerance to a dietary antigen derived from a food in a subject in need thereof.

11. A therapeutically effective amount of a peptide related to Cpn60.1 for use of claim 10, wherein the Cpn60.1-related peptide is selected from the group consisting of:
(SEQ ID NO: 1; '1104)
DGSVVVNKVSELPAGHGLNVNTLSYGDLAAD;
(SEQ ID NO: 2)
DGSVVVNKVSELPAGH;
(SEQ ID NO: 3)
GLNVNTLSYGDLAAD;
(SEQ ID NO: 4)
SELPAGHGLNVNLTS;
(SEQ ID NO: 5)
DGSVVVNKVS;
(SEQ ID NO: 6)
ELPAGHGLNV;
(SEQ ID NO: 7)
NTLSYGDLAAD;
and
or a functionally equivalent fragment or variant thereof.

12. A therapeutically effective amount of a peptide related to Cpn60.1 for use of claim 11, wherein the Cpn60.1-related peptide is SEQ ID NO:1.

13. A therapeutically effective amount of a peptide related to Cpn60.1 for use of any one of claims 10-12, further comprising, prior to the use, diagnosing the subject as having, or likely to develop, a food allergy or food intolerance, or receiving the results of an assay that diagnoses the subject as having, or likely to develop, a food allergy or food intolerance.

14. A therapeutically effective amount of a peptide related to Cpn60.1 for use of any one of claims 10-13, wherein the Cpn60.1-related peptide is used prior to the first exposure to a potential causal food allergen, or upon clinical signs of atopic symptoms.

15. A therapeutically effective amount of a peptide related to Cpn60.1 for use of any one of claims 10-14, wherein the subject has been diagnosed with at least one food allergy or food intolerance.

16. A therapeutically effective amount of a peptide related to Cpn60.1 for use of any one of claims 10-15, wherein the dietary antigen is derived from milk and products thereof; eggs and products thereof; meat and products thereof; fish, mollusks, and crustaceans and products thereof; oils, fats, and products thereof; grains and products thereof; pulses, seeds, kernels, nuts, and products thereof; vegetables and products thereof; fruits and products thereof; mushrooms and products thereof; sugar, sugar products, chocolate products, and confectionary; and spices and herbs.

17. A therapeutically effective amount of a peptide related to Cpn60.1 for use of any one of claims 10-16, wherein the use of the Cpn60.1-related peptide is acute or short-term.

18. A therapeutically effective amount of a peptide related to Cpn60.1 for use of any one of claims 10-16, wherein the use of the Cpn60.1-related peptide is long-term.
